SEQUENCE LISTING (25 sequences)

SEQ ID NO: 1 - protein, 13 aa, Agalychnis calcarifer
RRKPLFPFIPRPK

SEQ ID NO: 2 - protein, 12 aa, Agalychnis litodryas
MLADVFEKIMGD

SEQ ID NO: 3 - protein, 28 aa, Agalychnis litodryas
AVWKDFLKNIGKAAGKAVLNSVTDMVNE

SEQ ID NO: 4 - protein, 13 aa, Bombina variegata
QRLGHQWAVGHLM

SEQ ID NO: 5 - protein, 13 aa, Bombina variegata
DSFGNQWARGHFM

SEQ ID NO: 6 - protein, 14 aa, Bombina variegata
GKPFYPPPIYPEDM

SEQ ID NO: 7 - protein, 21 aa, Bombina variegata
IYNAICPCKHCNKCKPGLLAN

SEQ ID NO: 8 - protein, 13 aa, Phyllomedusa trinitatis
  misc_feature (1)..(2): Xaa can be any naturally occurring amino acid
XXPLAPFFQAVFK

SEQ ID NO: 9 - protein, 28 aa, Phyllomedusa trinitatis
ALWKDILKNVGKAAGKAVLNTVTDMVNQ

SEQ ID NO: 10 - protein, 27 aa, Rana palustris
ALSILRGLEKLAKMGIALTNCKATKKC

SEQ ID NO: 11 - protein, 24 aa, Rana pipiens
FLPIIAGVAAKVFPKIFCAISKKC

SEQ ID NO: 12 - protein, 19 aa, Rana saharica
KGAAKGLLEVASCKLSKSC

SEQ ID NO: 13 - protein, 46 aa, Rana saharica
GIFSKFGRKKIKNLLISGLKNVGKEVGMDVVRTGIDIAGCKIKGEC

SEQ ID NO: 14 - protein, 46 aa, Rana saharica
GIFSKLAGKKLKNLLISGLKNVGKEVGMDVVRTGIDIAGCKIKGEC

SEQ ID NO: 15 - protein, 33 aa, Rana saharica
GILSTIKDFAIKAGKGAAKGLLEMASCKLSGQC

SEQ ID NO: 16 - protein, 34 aa, Rana saharica
GILLDKLKNFAKTAGKGVLQSLLNTASCKLSGQC

SEQ ID NO: 17 - protein, 24 aa, Rana saharica
FLPLLAGLAANFLPKIFCKITRKC

SEQ ID NO: 18 - protein, 24 aa, Rana saharica
AVITGACERDVQCGGGTCCAVSLI

SEQ ID NO: 19 - protein, 23 aa, Agalychnis litodryas
AWKDLKNGKAAGKAVLNVTDMVN

SEQ ID NO: 20 - protein, 11 aa, Rana saharica
KGLLASCKLSC

SEQ ID NO: 21 - protein, 15 aa, Rana spp.
FLPAGAAPKIFCIKC

SEQ ID NO: 22 - protein, 42 aa, Rana saharica
GIFSKKKKNLLISGLKNVGKEVGMDVVRTGIDIAGCKIKGEC

SEQ ID NO: 23 - protein, 7 aa, Bombina variegata
GQWAGHM

SEQ ID NO: 24 - protein, 22 aa, Rana saharica
GILLKFAAGKGLLASCKLSGQC

SEQ ID NO: 25 - protein, 13 aa, Bombina variegata
QRLGNQWAVGHLM

(19) United States
(12) Patent Application Publication
(10) Pub. No.: US 2008/0058269 A1
(43) Pub. Date: Mar. 6, 2008

(54) Insulin Releasing Peptides

(75) Inventors: Yassar Hassan Atef Abdel-Wahab, Portrush (GB); Lamin Marenah, Portstewart (GB); David Francis Orr, Kilkeel (GB); Stephen McClean, Coleraine (GB); Peter Raymond Flatt, Portrush (GB); Chris Shaw, Comber (GB)

Correspondence Address: Patent Administrator;Kirkpatrick & Lockhart Nicholson, State Street Financial Center, One Lincoln Street, Boston, MA, 02111-2950, US

(73) Assignee: UNVERSITY OF ULSTER, COLERAINE, CO LONDONDERRY UNITED KINDGOM (GB)

(21) Appl. No.: 10/579,581

(22) PCT Filed: Nov. 17, 2004
(86) PCT No.: PCT/EP04/13693

§ 371 Date: Sep. 5, 2006

(30) Foreign Application Priority Data
Nov. 17, 2003 (GB) 0326720.0

(51) Int. Cl. A61K 38/10; A61K 38/17; A61P 7/12; C07K 14/46

(52) U.S. Cl. 514/13; 530/323

(57) ABSTRACT

The present invention relates to peptides, or fragments thereof, isolated from the skin secretions of amphibians and which act as stimulators of insulin secretion and pancreatic beta cell function. The invention also encompasses peptide having at least 80%, preferably at least 85%, more preferably at least 90%, optionally more than 95%, sequence identity based on the ClustalW alignment method with the respective amino acid sequences of SEQ ID Nos. 1 to 17. Furthermore, the invention also includes peptides selected from the group comprising brevinins, dermaseptins and esculentins for stimulating insulin secretion by activation of physiological stimulus-secretion coupling pathways. These peptides are useful to stimulate insulin secretion and/or moderate blood glucose excursions. These peptides may be used for treatment of type 1 or type 2 diabetes mellitus.

The present invention relates to the discovery of novel insulin-releasing peptides in the skin secretions of amphibians and their potential use as stimulators of pancreatic beta cell function in the treatment of diabetes mellitus.

Amphibian skin contains two kinds of secretory glands. Mucous glands are distributed throughout the body, and their secretions mainly provide the moist coating necessary for cutaneous respiration. The granular glands, also known as serous or poison glands may be distributed or concentrated in certain areas of the body. The secretions from the granular glands of anurans (frogs and toads) have been shown to contain pharmacologically active substances ranging from simple amines such as norepinephrine and histamine to biologically active peptides, piperidine and steroidal alkaloids, bufodienolides and tetrodotoxin [Bevins C L and Zasloff M 1990 Peptides from frog skin. Annual Review of Biochemistry 59 395-414; Erspamer V & Melchiorri P 1983 Neuroendocrine perspectives. pp. 37-104. Elsevier Science, Amsterdam]. These compounds are thought to play various roles, either in the regulation of physiological functions of the skin or in defence against predators or microorganisms [Barthalmus G T 1994 Amphibian biology. pp. 382-410. Beatty and Sons, Surrey.: Barra D and Simmaco M. 1995 Amphibian slin: a promising resource for antimicrobial peptides. Trends In Biotechnology 13 205-209].

During evolution some of these agents, or closely related daughter molecules, may have taken on distinct physiological roles in humans. It follows that pharmacologically active substances isolated from the skin secretions of frogs and toads may be useful for the treatment of human clinical disease such as diabetes mellitus.

The invention will now be demonstrated with reference to the following non-limiting examples and the accompanying figures wherein:

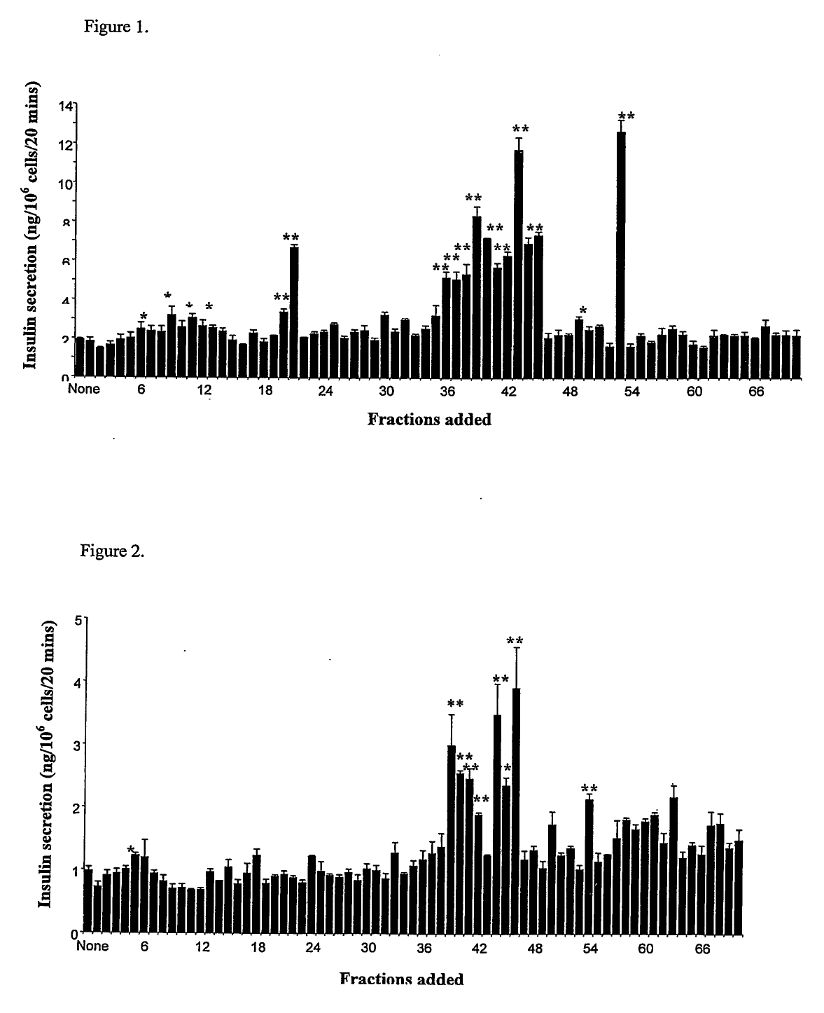

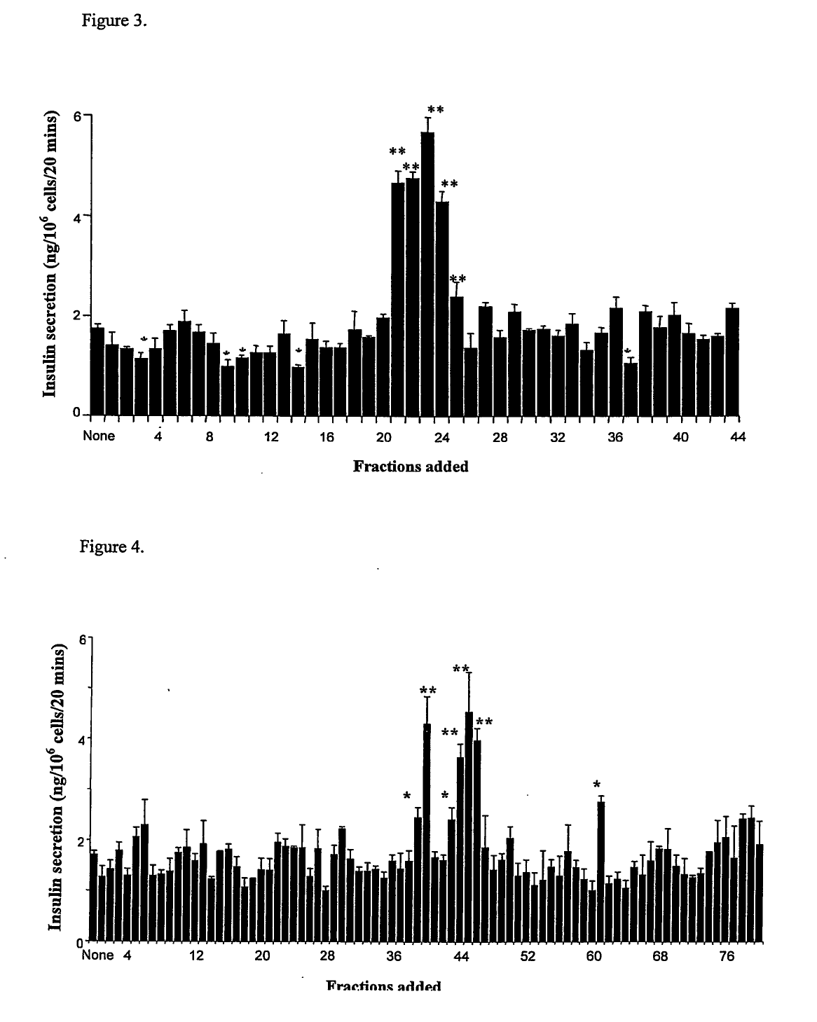

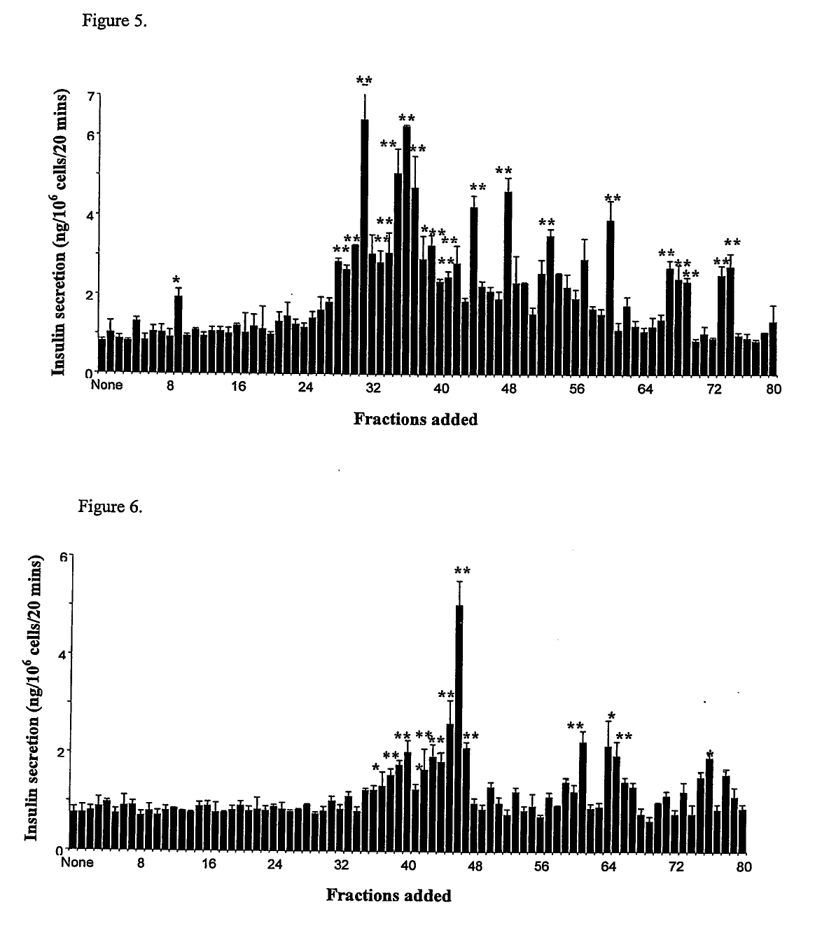

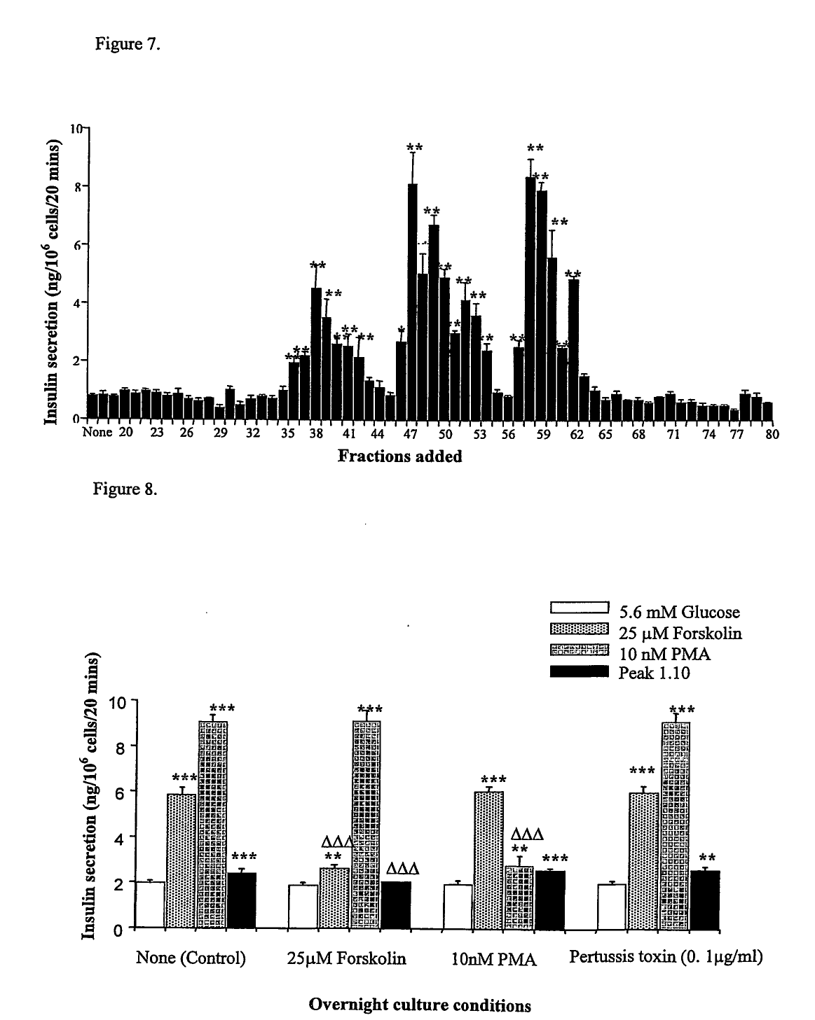

FIGS. 1-7 illustrate that semi-preparative C18 HPLC fractions of the skin secretions of various amphibians (Agalychnis calcarifer, Agalychnis litodryas, Bombina variegata, Phyllomedusa trinitatis, Rana palustris, Rana pipiens and Rana saharica, respectively) stimulate insulin secretion from BRIN-BD11 cells. Legend: Effects of various semi-preparative C18 HPLC fractions of the respective specified crude venom on insulin secretion from BRIN-BD11 cells. Incubations were performed at 5.6 mM glucose. Values are the mean ±SEM for 3 separate observations. *P<0.01 and **P<0.001 compared with 5.6 mM glucose alone.

FIG. 8 illustrates the dependence of the stimulatory effects of 1653.2 Da purified peptide (peak 1.10) from Agalychnis calcarifer on intracellular pathways mediated by protein kinase A (forskolin), protein kinase C (PMA) or pertussis toxin-sensitive G-proteins. Legend: Acute effects of the 13 amino acid 1653.2 Da purified peptide (peak 1.10) from Agalychnis calcarifer on forskolin and PMA on insulin secretion from BRIN-BD11 cells cultured overnight in the absence (control) and presence of 25 μM forskolin, 10 nM PMA or 0.1 μg/ml pertussis toxin. Acute incubations were performed at 5.6 mM glucose. Values are the mean ±SEM for 8 separate observations. ***P<0.001 compared with 5.6 mM glucose alone under same culture conditions. ^{ΔΔΔ}p<0.001 compared with respective test reagent following control culture.

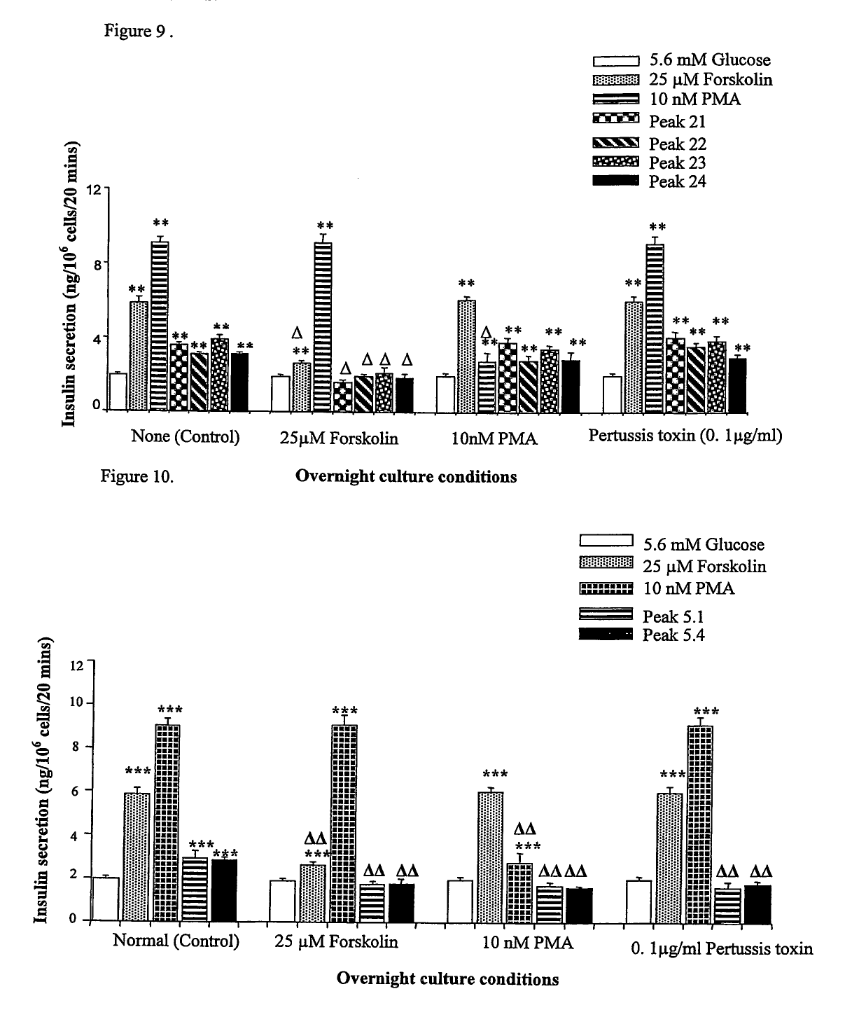

FIG. 9 illustrates the dependence of the stimulatory effects of 1641.7 Da, 1662.6 Da, 1619.8 Da and 1650.5 Da purified peptides (peaks 21, 22, 23 and 24) from Bombina variegata on intracellular pathways mediated by protein kinase A (forskolin), protein kinase C (PMA) or pertussis toxin-sensitive G-proteins. Legend: Acute effects of 1641.7, 1662.6, 1619.8 and 1650.5 Da purified peptides (peaks 21, 22, 23 and 24) from Bombina variegata on forskolin and PMA on insulin secretion from BRIN-BD11 cells cultured overnight in the absence (control) and presence of 25 μM forskolin, 10 nM PMA or 0.1 μg/ml pertussis toxin. Acute incubations were performed at 5.6 mM glucose. Values are the mean ±SEM for 8 separate observations. **P<0.001 compared with 5.6 mM glucose alone under same culture conditions. ^{Δ}P<0.001 compared with respective test reagent following control culture.

FIG. 10 illustrates the dependence of the stimulatory effects of 4920.4 Da and 4801.2 Da purified peptides (peaks 5.1 and 5.4) from Rana saharica on intracellular pathways mediated by protein kinase A (forskolin), protein kinase C (PMA) or pertussis toxin-sensitive G-proteins. Legend: Acute effects of 4920.4 and 4801.2 Da purified peptides (peaks 5.1 and 5.4) from Rana saharica on forskolin and PMA on insulin secretion from BRIN-BD11 cells cultured overnight in the absence (control) and presence of 25 μM forskolin, 10 nM PMA or 0.1 g/ml pertussis toxin. Acute incubations were performed at 5.6 mM glucose. Values are the mean ±SEM for 8 separate observations. ***P<0.001 compared with 5.6 mM glucose alone under same culture conditions. ^{ΔΔ}P<0.001 compared with respective test reagent following control culture.

According to a first aspect of the invention there are provided various peptides, and their fragments, as stimulators of insulin secretion and pancreatic beta cell function.

Sequennce nos. d and e show 62% sequence homology and sequence nos. c and i show 82% sequence homology. Sequence nos. m and n show 91% sequence homology and sequence nos. k and q show 67% sequence homology. Sequence nos. l, o and p show sequence homology—sequence nos. l and o show 84% sequence homology, o and p show 65% sequence homology and l and p show 58% sequence homology.

The ClustalW alignment data are given below:

CLUSTAL W (1.82) multiple sequence alignment

| | | |
|---|---|---|
| d | -------------------QRLGHQW-AVGHLM-------------- | 13 |
| e | -------------------DSFGNQW-ARGHFM-------------- | 13 |
| c | AVWKDF------------LKNIGKAA-GKAVLNSVTDMVNE------ | 28 |
| i | ALWKDI------------LKNVGKAA-GKAVLNTVTDMVNQ------ | 28 |
| m | GIFSKFGRKKIKNLLISGLKNVGKEV-GMDVVRTGIDIAGCKIKGEC | 46 |
| n | GIFSKLAGKKLKNLLISGLKNVGKEV-GMDVVRTGIDIAGCKIKGEC | 46 |
| l | ----------------------------KGAAKGLLEVASCKLSKSC | 19 |
| o | GILS-T------------IKDFAIKA-GKGAAKGLLEMASCKLSGQC | 33 |
| p | GILLDK------------LKNFAKTA-GKGVLQSLLNTASCKLSGQC | 34 |
| k | ------------------FLPIIAGV-AAKVFPKIF----CAISKKC | 24 |
| q | ------------------FLPLLAGL-AANFLPKIF----CKITRKC | 24 |
| b | -------------------------M-LADVFEKIM----GD----- | 12 |
| a | ----------------------RRKP-LFPFIPRPK----------- | 13 |
| | ----------------------GKPFYPPPIYPEDM----------- | 14 |
| h | ----------------------XXPL--APFFQAVFK---------- | 13 |
| g | -----------------IYNAICPCKHCNKCKPGLLAN--------- | 21 |
| j | ------------------ALSILRGL--EKLAKMGIALTNCKATKKC | 27 |

In a second aspect, the invention provides brevinins, dermaseptins and esculentins that stimulate insulin secretion by activation of physiological stimulus-secretion coupling pathways.

In each of the first and second aspects, the invention includes similar fragments and such similar fragments are characterised in the present invention, by % identity to the sequences disclosed herein, as determined by algorithms commonly known to those skilled in the art. For example, multiple alignment of sequences may be performed using the ClustalW method of alignment (Thompson et al, 1994, Nucleic Acids Research, 22, pp 4673-4680; Higgins & Sharp 1989 Cabios 5:151-153).

In a further aspect, the invention provides use of such peptides to stimulate insulin secretion and/or mid blood glucose excursions.

### EXAMPLE 1

Collection of skin secretions: Captive bred of Agalychnis calcarifer, Agalychnis litodryas, Bombina variegata, Phyllomedusa trinitatis, Rana palustris, Rana pipiens and Rana saharica were maintained in terraria at 24° C. with 12 h/12 h light/dark cycle and fed on crickets. The skin secretions were obtained from groups of four amphibians from each species by gentle electrical stimulation (4-ms pulse width, 50 Hz, 5 V) using platinum electrodes rubbed over the moistened dorsal skin surface for 10 s. Secretions were washed off into a glass beaker, using deionised water. The resultant secretions were freeze dried in a Hetosicc 2.5 freeze dryer (Heto, UK). Approximately 50 mg, dry weight, of skin secretion was obtained for each species.

Purification of peptide: Lyophilized crude venom (20 mg) from each species was dissolved in 0.12% trifluoroacetic acid/water (2 ml) and 1 ml of it was chromatographed on a Vydac 218TP510 semi-preparative C-18 column (25×1 cm, Hesperia, Calif., USA). The column was equilibrated with 0.12% (v/v) trifluoroacetic acid/water at a flow rate of 2 ml/min. Using 0.1% (v/v) TFA in 70% acetonitrile/water, concentration of acetonitrile in the eluting solvent was raised to 80% (v/v) over 80 min using linear gradients. Absorbance was monitored at 214 nm with collection of 2 ml fractions. Fractions which showed major insulin releasing activity were pooled and rechromatographed using a Vydac 208TP54 analytical C-18 column (25×0.46 cm). The column was equilibrated with 0.12% (v/v) trifluoroacetic acid/water at a flow rate of 1 ml/min. Using 0.1% (v/v) TFA in 70% acetonitrile/water, the concentration of acetonitrile in the eluting solvent was raised to 15% (v/v) over 5 min and to 80% (v/v) over 70 min using linear gradients. Absorbance was monitored at 214 mn.

Culture of insulin-secreting cells: Clonal rat insulin-secreting BRIN-BD11 cells were cultured in RPMI-1640 tissue culture medium containing 10% (v/v) fetal calf serum, 1% (v/v) antibiotics (100 U/ml penicillin, 0.1 mg/ml streptomycin) and 11.1 mM glucose. The production and characterisation of BRIN-BD11 cells are described elsewhere [5]. Cells were maintained in sterile tissue culture flasks (Corning, Glass Works, UK) at 37° C. in an atmosphere of 5% CO₂ and 95% air using LEEC incubator (Laboratory Technical Engineering, Nottingham, UK). In three experimental series using purified peptides from Agalychnis calcarifer, Bombina variegata and Rana saharica cells were cultured overnight with 25 μM forskolin, 10 nM PMA or 0.1 μg/ml pertussis toxin prior to acute tests.

Acute insulin release studies: Insulin release from BRIN-BD11 cells was determined using cell monolayers [McClenaghan N H, Barnett C R, Ah-Sing E, Abdel-Wahab Y H, O'Harte F P, Yoon T W, Swanston-Flatt S K and Flatt P R 1996 Characterization of a novel glucose-responsive insulin-secreting cell line, BRIN-BD11, produced by electrofusion. Diabetes 45 1132-1140]. The cells were harvested with the aid of trypsin/EDTA (Gibco), seeded into 24-multiwell plates (Nunc, Rosklide, Denmark) at a density of 1.5×10⁶ cells per well, and allowed to attach overnight. Prior to acute test, cells were preincubated for 40 min at 37° C. in a 1.0 ml Krebs Ringer bicarbonate buffer (115 mM NaCl, 4.7 mM KCl, 1.28 MM CaCl₂, 1.2 MM KH₂PO₄, 1.2 mM MgSO₄, 10 mM NaHCO₃, 5 g/l bovine serum albumin, pH 7.4) supplemented with 1.1 mM glucose. Test incubations were performed for 20 min at 37° C. using the same buffer supplemented with 5.6 mM glucose in the absence (control) and presence of various venom fractions, peaks (equivalent to approx. 25 μl dried HPLC fraction) or test agents as indicated in the Figures. Cell viability after 20 min test incubations was assessed by modified neutral red assay [Hunt S M, Chrzanowska C, Barnnett C R, Brand H N and Fawell J K 1987 A comparison of in vitro cytotoxicity assays and their application to water samples. Alternatives to Laboratory Animals 15 20-29]. After incubation, aliquots of buffer were removed and stored at −20° C. for insulin radiomnimmunoassay [Flatt P R. and Bailey C J 1981 Abnormal plasma glucose and insulin responses in heterozygous lean (ob/+) mice. Diabetologia 20 573-577].

Molecular mass determination: The molecular masses of the purified individual non-toxic peaks exhibiting insulin releasing activity were determined using Matrix Assisted Laser Desorption Ionisation-Time of Flight (MALDI-TOF) mass spectrometry. Electrospray Ionisation quadripole ion-trap Mass Spectrometry (ESI-MS) was used for Agalychnis calcarifer, Bombina variegata, Rana pipiens and Rana saharica. Masses were recorded and compared with theoretical values calculated by the peptide calculator, a computer software package.

Depyroglutamation: Where necessary (Bombina variegata), pyroglutamate at the N-terminal was removed by adding 25 μl of pyroglutamnate aminopeptidase preparation (50 mM Na₂HPO₄, 10 mM β-mercaptoethanol, 1 mM dithiothreitol, and 1 mM EDTA adjusted to pH 7.3 with H₃HPO₄) containing 0.4 mg/ml pyroglutamate aminopeptidase to 100 μl of the lypophilised peptide. The reaction mix was incubated for 2 hours at 37° C. and then stored at −20° C. for subsequent amino acid determination by Edman degradation.

Structural analysis by automated Edman degradation: The primary structures of the purified peptides were determined by automated Edman degradation, using an Applied Biosystems Procise 491 microsequencer. Standard operating procedures were used (Applied Biosystems Model 491 Protein Sequencers Users Manual). The limit for detection of phenylthiohydantoin amino acids was 0.2 pmol. The primary structures were compared with those deposited in the SWISSPROT™ database.

Statistical analysis: Results are expressed as mean ±S.E.M. Values were compared using Student's unpaired t-test. Groups of data were considered to be significantly different if P<0.05.

### Results

Isolation, mass spectrometry and sequence analysis of insulin-releasing peptides: Skin secretions from the various amphibian species were purified by HPLC, yielding in each case multiple fractions that were subsequently screened for in vitro biological activity using BRIN-BD11 cells. The insulin-releasing profiles of peaks emerging from the primary HPLC separation are illustrated for Agalychnis calcarifer, Agalychnis litodryas, Bombina variegata, Phyllomedusa trinitatis, Rana palustris, Rana pipieizs and Rana saharica in FIGS. 1-7, respectively.

The major peaks of insulin-releasing activity were subjected to further HPLC purification steps, giving rise ultimately to the isolation of pure peptides with proven insulinotropic activity (Tables 1-7). Where sufficient sample was available, molecular masses and either partial or complete sequences were determined for each peptide as summarised for the various amphibian species in Tables 1-7. In instances where a complete sequence was obtained, the theoretical (calculated) molecular masses of the peptides were shown to corresponded closely to the experimental masses. This indicates the absence of any post-translational modification of constituent amino acids, such as phosphorylation, sulphation or glycation.

TABLE 1
Insulin secretion, experimental molecular mass, amino acid sequence,
database comparison and theoretical molecular mass of individual peptides isolated
from Agalychnis calcarifer.

| | | | | | Theoretical |
|---|---|---|---|---|---|
| Peptide | Insulin release | Experimental | Amino acid | Database | (Calculated) |
| ID | (ng/10⁶ cells/20 mins) | Mass (Da) | sequence | match | Mass (Da) |
| None | 1.71 ± 0.12 | | | | |
| 1.3 | 2.52 ± 0.23** | ND | No sequence | | |
| 1.10 | 2.61 ± 0.11** | 1653.2 | RRKPLFPFIPRPK | No | 1652.1 |
| | | | | Match | |
| 1.17 | 2.10 ± 0.15* | ND | No sequence | | |
| 1.18 | 2.52 ± 0.05*** | ND | No sequence | | |

TABLE 2
Insulin secretion, experimental molecular mass, amino acid sequence,
database comparison and theoretical molecular mass of individual peptides isolated
from Agalychnis litodryas.

| | | | | | Theoretical |
|---|---|---|---|---|---|
| Peptide | Insulin release | Experimental | Amino acid | Database | (Calculated) |
| ID | (ng/10⁶ cells/20 mins) | Mass (Da) | sequence | match | Mass (Da) |
| None | 1.95 ± 0.16 | | | | |
| 1.7 | 4.22 ± 0.48*** | 2546.2 | MLADVFEKIMGD . . . | | |
| | | | (Insufficient | | |
| | | | sample) | | |
| 2.9 | 7.46 ± 0.08*** | 3020.0 | AVWKDFLKNIGK | Dermasept | 3019.5 |
| | | | AAGKAVLNSVTD | in B IV | |
| | | | MVNE | precursor | |
| | | | | 79% ID | |

Incubations were performed at 5.6 mM glucose.
Values are mean ± SEM for 3 separate observations.
**P < 0.01 and
***P < 0.001 compared with 5.6 mM glucose.
ND = Not detected.
Single letter code denote amino acids: A, Ala; R, Arg; N, Asn; D, Asp; C, Cys; E, Glu; Q, Gln; G, Gly; H, His; X, Hyp; I, Ile; L, Leu; K, Lys; M, Met; F, Phe; P, Pro; S, Ser; T, Thr; W, Trp; Y, Tyr; V, Val

TABLE 3
Insulin secretion, experimental molecular mass, amino acid sequence,
database comparison and theoretical molecular mass of individual peptides isolated
from Bombina variegata.

| | | | | | Theoretical |
|---|---|---|---|---|---|
| | Insulin release | Experimental | Amino acid | | (Calculated) |
| Peptide ID | (ng/10⁶ cells/20 mins) | Mass (Da) | sequence | Database match | Mass (Da) |
| None | 1.74 ± 0.08 | | | | |
| 21 | 4.66 ± 0.24*** | 1641.7 | Pyr- | Bombesin | 1642.7 |
| | | | QRLGHQWAVG | 93% ID | |
| | | | HLM-amidated | (His)⁶Bombesin | |
| 22 | 4.75 ± 0.13*** | 1662.6 | Pyr- | Bombesin | 1662.9 |
| | | | DSFGNQWARG | 72% ID | |
| | | | HFM-amidated | | |
| 23 | 5.67 ± 0.30*** | 1619.8 | Pyr- | Bombesin | 1620.7 |
| | | | QRLGNQWAVG | 100% ID | |
| | | | HLM-amidated | | |
| 24 | 4.30 ± 0.20*** | 1650.5 | GKPFYPPPIYPE | Tryptophyllin | 1650.9 |
| | | | DM | 57% ID | |
| 25 | 2.39 ± 0.30*** | 2300.0 | IYNAICPCKHCN | No | 2299.8 |
| | | | KCKPGLLAN | Match | |

TABLE 4
Insulin secretion, experimental molecular mass, amino acid sequence,
database comparison and theoretical molecular mass of individual peptides isolated
from Phyllomedusa trinitatis.

| | | | | | Theoretical |
|---|---|---|---|---|---|
| Peptide | Insulin release | Experimental | | Database | (Calculated) |
| ID | (ng/10⁶ cells/20 mins) | Mass (Da) | Amino acid sequence | Match | Mass (Da) |
| None | 1.47 ± 0.04 | | | | |
| 1.8 | 2.48 ± 0.37** | 8326.4 | XXPLAPFFQAVFK . . . | | |
| | | | (Insufficient sample) | | |
| 1.11 | 2.10 ± 0.16** | 3379.9 | ND | | |
| 2.10 | 2.356 ± 0.34** | 2996.4 | ALWKDILKNVGKA | Dermaseptin | 2998.5 |
| | | | AGKAVLNTVTDMV | B IV | |
| | | | NQ | precursor | |
| | | | | 100% ID | |

TABLE 5
Insulin secretion, experimental molecular mass, amino acid sequence,
database comparison and theoretical molecular mass of individual peptides isolated
from Rana palustris.

| | | | | | Theoretical |
|---|---|---|---|---|---|
| Peptide | Insulin release | Experimental | | Database | (Calculated) |
| ID | (ng/10⁶ cells/20 mins) | Mass (Da) | Amino acid sequence | match | Mass (Da) |
| None | 1.467 ± 0.04 | | | | |
| 2.6 | 1.93 ± 0.23** | ND | ND | | |
| 2.7 | 4.14 ± 0.40*** | 8560.4 | ND | | |
| 3.1 | 2.12 ± 0.09*** | 4919.9 | ND | | |
| 3.8 | 2.48 ± 0.44** | 2873.5 | ALSILRGLEKLAK | Brevinin-1 | 2873.7 |
| | | | MGIALTNCKATKKC | (46% ID) | |
| 4.3 | 2.14 ± 0.13*** | 3848.7 | ND | | |
| 4.4 | 1.87 ± 0.06** | ND | ND | | |

TABLE 6
Insulin secretion, experimental molecular mass, amino acid sequence,
database comparison and theoretical molecular mass of individual peptides isolated
from Rana pipiens.

| | | | | | Theoretical |
|---|---|---|---|---|---|
| Peptide | Insulin release | Experimental | Amino acid | | (Calculated) |
| ID | (ng/10⁶ cells/20 mins) | Mass (Da) | sequence | Database match | Mass (Da) |
| None | 2.76 ± 0.13 | | | | |
| 3.1 | 3.46 ± 0.17*** | 5125.2 | ND | | |
| 4.1 | 4.15 ± 0.01*** | 2562.6 | FLPIIAGVAAKV | Pipinin-1 | 2563.2 |
| | | | FPKIFCAISKKC | 100% ID) | |

Incubations were performed at 5.6 mM glucose.
Values are mean ± SEM for 3 separate observations.
**P < 0.01 and
***P < 0.001 compared with 5.6 mM glucose.
ND = Not detected.
Single letter code denote amino acids: A, Ala; R, Arg; N, Asn; D, Asp; C, Cys; E, Glu; Q, Gln; G, Gly; H, His; X, Hyp; I, Ile; L, Leu; K, Lys; M, Met; F, Phe; P, Pro; S, Ser; T, Thr; W, Trp; Y, Tyr; V, Val

TABLE 7
Insulin secretion, experimental molecular mass, amino acid sequence,
database comparison and theoretical molecular mass of individual peptides isolated
from Rana saharica.

| | | | | | Theoretical |
|---|---|---|---|---|---|
| Peptide | Insulin release | Experimental | | Database | (Calculated) |
| ID | (ng/10⁶ cells/20 mins) | Mass (Da) | Amino acid sequence | match | Mass (Da) |
| None | 1.87 ± 0.06 | | | | |
| 4.14 | 3.15 ± 0.23** | ND | ND | | |
| 4.18 | 3.52 ± 0.21*** | ND | ND | | |
| 4.22 | 3.47 ± 0.40*** | 1892.6 | KGAAKGLLEVASC | Rugosin A | 1891.2 |
| | | | KLSKSC | 68.4% ID | |
| 4.23 | 4.25 ± 0.17*** | 2930.8 | AVITGACERDVQC | Protein | 2322.6 |
| | | | GGGTCCAVSLI . . . | A/BV8 | |
| | | | (insufficient sample) | 78% ID | |
| 4.26 | 3.08 ± 0.19** | 1433.7 | ND | | |
| 4.27 | 3.09 ± 0.23** | ND | ND | | |
| 4.28 | 3.19 ± 0.08*** | ND | ND | | |
| 5.1 | 3.32 ± 0.22*** | 4920.4 | GIFSKFGRKKIKNL | Esculentin-1 | 4919.2 |
| | | | LISGLKNVGKEVG | 98% ID | |
| | | | MDVVRTGIDIAGC | | |
| | | | KIKGEC | | |
| 5.2 | 2.95 ± 0.08*** | 3404.6 | ND | | |
| 5.3 | 2.54 ± 0.15** | ND | ND | | |
| 5.4 | 3.30 ± 0.22*** | 4801.2 | GIFSKLAGKKLKN | Esculentin- | 4800.8 |
| | | | LLISGLKNVGKEV | 1B | |
| | | | GMDVVRTGIDIAG | 100% ID | |
| | | | CKIKGEC | | |
| 5.6 | 2.86 ± 0.37** | 3309.2 | GILSTIKDFAIKAG | Brevinin- | 3309.0 |
| | | | KGAAKGLLEMASC | 2EB | |
| | | | KLSGQC | 67% ID | |
| 6.5 | 5.93 ± 0.47*** | 3519.3 | GILLDKLKNFAKT | Brevinin- | 3519.2 |
| | | | AGKGVLQSLLNTA | 2EC | |
| | | | SCKLSGQC | 100% ID | |
| 6.7 | 3.46 ± 0.28** | 3119.2 | ND | | |
| 8.3 | 3.53 ± 0.06*** | 2676.9 | FLPLLAGLAANFLP | Brevinin- | 2676.4 |
| | | | KIFCKITRKC | 1E | |
| | | | | 100% ID | |

Incubations were performed at 5.6 mM glucose.
Values are mean ± SEM for 3 separate observations.
**P < 0.01 and
***P < 0.001 compared with 5.6 mM glucose.
ND = Not detected.
Single letter code denote amino acids: A, Ala; R, Arg; N, Asn; D, Asp; C, Cys; E, Glu; Q, Gln; G, Gly; H, His; X, Hyp; I, Ile; L, Leu; K, Lys; M, Met; F, Phe; P, Pro; S, Ser; T, Thr; W, Trp; Y, Tyr; V, Val

Six of the insulinotropic peptides have proved to be established structures. Peak 4.1 from Rana pipiens was identical to pipinin-1, peak 23 from Bombina variegata to bombesin, peak 2.10 from Phyllomedusa trinitatis to dermaseptin BIV precursor and peaks 5.4, 6.5 and 8.3 from Rana saharica matched Esculentin-1B, Brevinin-2EC and Brevinin-1E respectively. With these few exceptions, all other insulin-releasing peptides were novel structures as established using the SWISSPROT™ database. Even the functional observations with pipinin-1, dermaseptin BIV precursor esculentin-1B and brevinin-2EC and 1E were novel as these were totally unsuspected insulin releasing peptides.

Modest similarity existed between some of the isolated insulin-releasing peptides and amphibian antimicrobial peptides that are unsuspected insulin secretagogues such as brevinin, dermaseptin, Rugosin A and tryptophyllin. However, unlike the latter agents, no evidence was obtained of cell lysis or a toxic action that might account for insulin secretion. Thus the peptides reported herein appear to act through physiological mammalian processes controlling exocytosis of insulin.

To support this view, further studies were carried out using purified peptides from Agalychins calcarifer, Bombina variegata and Rana saharica to examine cellular mechanisms underlying the stimulation of insulin secretion. The stimulatory effects of the 1653.2 Dapeptide (peak 1.10) from Agalychnis calcarifer (FIG. 8) and 1641.7, 1662.6, 1619.8 and 1650.5 Dapeptides (peaks 21, 22, 23 and 24 respectively) from Bombina variegata (FIG. 9) were abolished in cells cultured overnight with forskolin to desensitise the cyclic AMP-protein kinase A pathway. Overnight culture with PMA or pertussis toxin did not affect the insulin-releasing ability of the peptides, suggesting lack of involvement of protein kinase C or G-protein dependent pathways. Overnight culture with forskolin or PMA resulted in the abolition of the acute stimulatory effects of forskolin or PMA, respectively (FIGS. 8 and 9). Interestingly, the insulin-releasing action of the 1653.2 Da peptide from Agalychnis calcarifer and peaks 21, 22, 23 and 24 from Bombina variegata were not affected by 50 μM verapamil and were clearly evident in cells depolarised with 30 mM KCl (Tables 8 and 9).

TABLE 8
Effects of the 13 amino acid peptide peak (peak 1.10) from
Agalychnis calcarifer on insulin secretion from BRIN-BD11 cells
in the presence of verapamil or a depolarising K⁺ concentration.

Insulin secretion (ng/10⁶ cells/20 min)

| Addition | Control | Peak 1.10 |
|---|---|---|
| None | 1.68 ± 0.16 | 2.75 ± 0.12 * |
| Verapamil (50 μM) | 1.74 ± 0.17 | 3.54 ± 0.20 ΔΔ*** |
| KCl (30 mM) | 5.32 + 0.38 ΔΔΔ | 16.45 ± 0.10 ΔΔ*** |

TABLE 9
Effects of peaks 21, 22, 23 and 24 from Bombina variegata on insulin
secretion from BRIN-BD11 cells in the presence of verapamil or a depolarising K⁺
concentration.

| | Insulin secretion (ng/10⁶ cells/20 min) | | | | |
|---|---|---|---|---|---|
| Addition | Control | Peak 21 | Peak 22 | Peak 23 | Peak 24 |
| None | 1.69 ± 0.17 | 3.65 ± 0.05*** | 3.23 ± 0.14*** | 3.40 ± 0.14*** | 3.31 ± 0.20*** |
| Verapamil | 1.74 ± 0.17 | 4.25 ± 0.82*** | 3.05 ± 0.09*** | 3.21 ± 0.14*** | 3.08 ± 0.29*** |
| (50 μM) | | | | | |
| KCl (30 mM) | 5.33 ± 0.38ΔΔΔ | 13.68 ± 1.42ΔΔΔ*** | 13.69 ± 1.43ΔΔΔ*** | 15.21 ± 1.43ΔΔΔ*** | 15.35 ± 0.41ΔΔΔ*** |

Acute incubations were performed at 5.6 mM glucose.
Values are mean ± SEM for 8 separate observations.
*P < 0.05 and
***P < 0.001 compared with control,
ΔΔP < 0.01 and
ΔΔΔP < 0.001 compared with no addition.

The stimulatory effects of the 4920.4 and 4801.2 Da peptides (peaks 5.1 and 5.4) from Rana saharica were abolished in cells cultured overnight with forskolin, PMA or pertussis toxin (FIG. 10) indicating the involvement of both protein kinase A and C and pertussis toxin-sensitive G-protein in their stimulatory actions. As shown in Table 10, the insulin releasing actions of the isolated peptides were not inhibited by the calcium channel blocker veraparnil. Stimulatory effects on insulin secretion were also clearly evident in cells depolarised by 30 mM KCl.

TABLE 10
Effects of peaks 5.1 and 5.4 from Rana saharica
on insulin secretion from BRIN-BD11 cells in the presence
of verapamil or a depolarising K⁺ concentration.

| | Insulin secretion (ng/10⁶ cells/20 min) | | |
|---|---|---|---|
| Addition | Control | Peak 5.1 | Peak 5.4 |
| None | 1.69 ± | 2.96 ± 0.32*** | 2.89 ± 0.19*** |
| | 0.17 | | |
| Verapamil | 1.74 ± | 2.88 ± 0.28*** | 2.79 ± 0.10*** |
| (50 μM) | 0.17 | | |
| KCl | 5.33 ± | 10.11 ± 0.81ΔΔΔ*** | 11.84 ± 0.98ΔΔΔ*** |
| (30 mM) | 0.38ΔΔΔ | | |

Acute incubations were performed at 5.6 mM glucose.
Values are mean ± SEM for 8 separate observations.
***P < 0.001 compared with control,
ΔΔΔP < 0.001 compared with no addition.

### Discussion

This research describes for the first time the isolation and characterisation of peptides with insulin-releasing activity from the skin secretions of Agalychnis calcarifer, Agalychnis litodryas, Bombina variegala, Phyllomedusa trinitatis, Rana palustris, Rana pipiens and Rana saharica. It is notable that this work has not only uncovered a diverse range of novel peptides structures but it has also revealed that the skin secretions from each amphibian species studied represents an unsuspected and rich source of peptides capable of stimulating physiological insulin secretion from mammalian pancreatic beta cells.

The insulin output induced by amphibian peptides is approximately equivalent to that induced by established mammalian gut peptides, GLP-1, GIP or CCK-8 [Gault V A, O'Harte F P M, Harriott P, Mooney M H, Green, B D and Flatt P R 2003 Effects of the novel (Pro³) GIP antagonist and extending (9-39) amide on GIP- and GLP-1-induced cyclic AMP generation, insulin secretion and postprandial insulin release in obese diabetic (ob/ob) mice: evidence that GIP is the major physiological incretin. Diabetologia 46 222-230; O'Harte F P M, Abdel-Wahab Y H, Conlon J M and Flatt P R 1998 Glycation of glucagon-like peptide-1(7-36)amide: characterization and impaired action on rat insulin secreting cells. Diabetologia 41 1187-1193; Abdel-Wahab Y H, O'Harte F P M, Mooney M H, Conlon J M and Platt P R 1999 N-terminal glycation of cholecystokinin-8 abolishes its insulinotropic action on clonal pancreatic B-cells. Biochimica et Biophysica Acta 1452 60-67]. This indicates that the amphibian peptides isolated are at least as capable as physiological mammalian hormones in stimulating insulin secretion. It is also clear that these peptides may also trigger insulin secretion and have other beneficial actions on beta cells which involve novel secretory pathways as suggested by studies using peak 1.10 from Agalychnis calcarifer and peaks 21, 22, 23 and 24 from Bombina variegata. In these cases the secretagogues appeared to be mediated through both protein kinase A and G-protein independent pathways. In the case of peptides isolated from Rana saharica (peaks 5.1 and 5.4), the stimulatory effects were also independent of pathways triggered by protein kinase C.

It is apparent from the insulin stimulatory effects that specific receptors must exist for these amphibian peptides on mammalian insulin-secreting beta cells. This gives rise to two major and highly novel non-exclusive possibilities. The first is that these insulin-releasing amphibian peptides have homologous or closely related mammalian counter-parts.

The second important possibility arising from this research is that the novel amphibian peptides described in Tables 1-7, or fragments thereof may offer a therapeutically useful means of treating insulin secretory dysfunction and other beta cell disturbances typical of diabetes in humans. Diabetes is predicted to reach epidemic proportions throughout the world in the next 20 years and current treatments do not restore normal glucose homeostasis, therein resulting in debilitating diabetic complications and premature death. Amphibian peptides may therefore be a useful addition to the therapeutic arsenal for use either alone or in combination with other agents to improve diabetes control and decrease the risk of associated complications.

### Peptide Sequences

<120>Title:
<130>AppFileReference:
<140>CurrentAppNumber:
<141>CurrentFilingDate:

### Sequence

<213>OrganismName: Agalychnis calcarifer
<400>PreSequenceString:
RRKPLFPFIP RPK 13
<212>Type: PRT
<211>Length: 13

SequenceName: 1 (formerly a)

SequenceDescription:

### Sequence

<213>OrganismName: Agalychnis litodryas
<400>PreSequenceString:
MLADVFEKIM GD 12
<212>Type: PRT
<211>Length: 12
SequenceName: 2 (formerly b)
SequenceDescription:

### Sequence

<213>OrganismName: Agalychnis litodryas
<400>PreSequenceString:
AVWKDFLKNI GKAAGKAVLN SVTDMVNE 28
<212>Type: PRT
<211>Length: 28

SequenceName: 3 (formerly c)

SequenceDescription:

### Sequence

<213>OrganismName: Bombina variegata
<400>PreSequenceString:
QRLGHQWAVGHLM 13
<212>Type: PRT
<211>Length: 13

SequenceName: 4 (formerly d)

SequenceDescription:

### Sequence

<213>OrganismName: Bombina variegata
<400>PreSequenceString:
DSFGNQWARGHFM 13
<212>Type: PRT
<211>Length: 13

SequenceName: 5 (formerly e)

SequenceDescription:

### Sequence

<213>OrganismName: Bombina variegata
<400>PreSequenceString:
GKPFYPPPIY PEDM 14
<212>Type: PRT
<211>Length: 14

SequenceName: 6 (formerly f)

SequenceDescription:

### Sequence

<213>OrganismName: Bombina variegata
<400>PreSequenceString:
IYNAICPCKH CNKCKPGLLA N 21
<212>Type: PRT
<211>Length: 21

SequenceName: 7 (formerly g)

SequenceDescription:

### Sequence

<213>OrganismName: Phyllomedusa trinitatis
<400>PreSequenceString:
XXPLAPFFQA VFK 13
<212>Type: PRT
<211>Length: 13

SequenceName: 8 (formerly h)

SequenceDescription:

### Sequence

<213>OrganismName: Phyllomedusa trinitatis
<400>PreSequenceString:
ALWKDILKNV GKAAGKAVLN TVTDMVNQ 28
<212>Type: PRT
<211>Length: 28

SequenceName: 9 (formerly i)

SequenceDescription:

### Sequence

<213>Organismname: Rana palustris
<400>PreSequenceString:
ALSILRGLEK LAKMGIALTN CKATKKC 27
<212>Type: PRT
<211>Length: 27

SequenceName: 10 (formerly j)

SequenceDescription:

### Sequence

<213>OrganismName: Rana pipiens
<400>PreSequenceString:
FLPIIAGVAA KVFPKIFCAI SKKC 24
<212>Type: PRT
<211>Length: 24

SequenceName: 11 (formerly k)

SequenceDescription:

### Sequence

<213>OrganismName: Rana saharica
<400>PreSequenceString:
KGAAKGLLEV ASCKLSKSC 19
<212>Type: PRT
<211>Length: 19

SequenceName: 12 (formerly l)

SequenceDescription:

### Sequence

<213>OrganismName: Rana saharica
<400>PreSequenceString:
GIFSKFGRKK IKNLLISGLK NVGKEVGMDV VRTGIDIAGC KIKGEC 46
<212>Type: PRT
<211>Length: 46

SequenceName: 13 (formerly m)

SequenceDescription:

### Sequence

<213>OrganismName: Rana saharica
<400>PreSequenceString:
GIFSKLAGKK LKNLLISGLK NVGKEVGMDV VRTGIDIAGC KIKGEC 46
<212>Type: PRT
<211>Length: 46

SequenceName: 14 (foemerly n)

SequenceDescription:

### Sequence

<213>OrganismName: Rana saharica
<400>PreSequenceString:
GILSTIKDFA IKAGKGAAKG LLEMASCKLS GQC 33
<212>Type: PRT
<211>Length: 33

SequenceName: 15 (formerly o)

SequenceDescription:

### Sequence

<213>OrganismName: Rana saharica
<400>PreSequenceString:
GILLDKLKNF AKTAGKGVLQ SLLNTASCKL SGQC 34
<212>Type: PRT
<211>Length: 34

SequenceName: 16 (formerly p)

SequenceDescription:

### Sequence

<213>OrganismName: Rana saharica
<400>PreSequenceString:
FLPLLAGLAA NFLPKIFCKI TRKC 24
<212>Type: PRT
<211>Length: 24

SequenceName: 17 (formerly q)

SequenceDescription:

1-9. (canceled)

10. Peptides, or fragments thereof, as stimulators of insulin secretion and pancreatic beta cell function, each peptide having at least 50% sequence identity based on the ClustalW alignment method with sequences selected from the group comprising AVITGACERDVQCGGGTCCAVSLI (SEQ ID NO:18) and the respective amino acid sequences of SEQ ID Nos. 1 to 17, each peptide being selected from the group comprising:
a peptide having at least 50% sequence identity based on the ClustalW alignment method with the amino acid sequence of dermaseptin and the partial structure A-WKD-LKN-GKAAGKAVLN-VTDMVN-(SEQ ID NO:19)
a peptide having at least 50% sequence identity based on the ClustalW alignment method with a peptide having a structure selected from the group comprising the partial structure KG-LL-ASCKLS-C (SEQ ID NO:20), the structure SEQ ID No. 6 GKPFYPPPIYPEDM (peptide 24, Bombina variegata), the partial structure FLP-AG-AA-PKIFC-I-KC (SEQ ID NO:21) and SEQ ID No. 10 ALSILRGLEKLAKMGIALTNCKATKKC (peptide 3.8, Rana palustris);
a peptide having at least 50% sequence identity based on the ClustalW alignment method with the amino acid sequence of esculentin and the partial structure GIFSK-KK-KNLLISGLKNVGKEVGMDVVRTGIDIAGCKIKGEC (SEQ ID NO:22);
a peptide having at least 50% sequence identity based on the ClustalW alignment method with the amino acid sequence of bombesin and the peptide having the partial structure -G-QWA-GH-M (SEQ ID NO:23); and
a peptide having at least 50% sequence identity based on the ClustalW alignment method with the amino acid sequence of a peptide selected from the group comprising AVITGACERDVQCGGGTCCAVSLI (SEQ ID NO:18), SEQ ID No. 1 RRKPLFPFIPRPK (peptide 1.10, Agalychnis calcarifer), SEQ ID No.7 IYNAICPCKHCNKCKPGLLAN (peptide 25, Bombina variegata), SEQ ID No. 2 a peptide having the N-terminus sequence MLADVFEKIMGD . . . (N-terminus of peptide 1.7, Agalychnis litodryas) and SEQ ID No. 8 a peptide having the N-terminus sequence XXPLAPFFQAVFK . . . (N-terminus of peptide 1.8, Phyllomedusa trinitatis).

11. A peptide according to claim 10, in which each peptide has at least 80% sequence identity based on the ClustalW alignment method with sequences selected from the group comprising AVITGACERDVQCGGGTCCAVSLI (SEQ ID NO:18) and the respective amino acid sequences of SEQ ID Nos. 1 to 17.

12. A peptide according to claim 10, in which each peptide has at least 90% sequence identity based on the ClustalW alignment method with sequences selected from the group comprising AVITGACERDVQCGGGTCCAVSLI (SEQ ID NO:18)and the respective amino acid sequences of SEQ ID Nos. 1 to 17.

13. A peptide according to claim 10, in which each peptide has more than 95% sequence identity based on the ClustalW alignment method with sequences selected from the group comprising AVITGACERDVQCGGGTCCAVSLI (SEQ ID NO: 18) and the respective amino acid sequences of SEQ ID Nos. 1 to 17.

14. A peptide according to claim 10, in which the peptide is selected from SEQ ID No. 3 AVWKDFLKNIGKAAGKAVLNSVTDMVNE (peptide 2.9, Agalychnis litodryas) and SEQ ID No. 9 ALWKDILKNVGKAAGKAVLNTVTDMVNQ (peptide 2.10, Phyllomedusa trinitatis).

15. A peptide according to claim 10, in which the partial structure is GIL-LK-FA-AGKG-LL-ASCKLSGQC (SEQ ID NO:24).

16. A peptide according to claim 15, in which the peptide is selected from SEQ ID No. 12 KGAAKGLLEVASCKLSKSC (peptide 4.22, Rana saharica), SEQ ID No. 15 GILSTIKDFAIKAGKGAAKGLLEMASCKLSGQC (peptide 5.6, Rana saharica) and SEQ ID No. 16 GILLDKLKNFAKTAGKGVLQSLLNTASCKLSGQC (peptide 6.5, Rana saharica).

17. A peptide according to claim 10, in which the peptide is selected from the group comprising SEQ ID No. 11 FLPIIAGVAAKVFPKIFCAISKKC (peptide 4.1, Rana pipiens) and SEQ ID No. 17 FLPLLAGLAANFLPKIFCKITRKC (peptide 8.3, Rana saharica).

18. A peptide according to claim 10, in which the peptide has the partial structure GIFSK-KK-KNLLISGLKNVGKEVGMDVVRTGIDIAGCKIKGEC (SEQ ID NO:22), the peptide being selected from SEQ ID No. 13 GIFSKFGRKKIKNLLISGLKNVGKEVGMDVVRTGIDIAGCKIKGEC (peptide 5.1 Rana saharica) and SEQ ID No. 14 GIFSKLAGKKLKNLLISGLKNVGKEVGMDVVRTGIDIAGCKIKGEC (peptide 5.4 Rana saharica).

19. A peptide according to claim 10, in which the partial structure is -G-QWA-GH-M (SEQ ID NO:23), the peptide being selected from the group comprising SEQ ID No. 4 Pyr-QRLGHQWAVGHLM-amidated (peptide 21, Bombina variegata) and SEQ ID No. 5 Pyr-DSFGNQWARGHFM-amidated (peptide 22, Bombina variegata).

20. A peptide as claimed in claim 10 with at least one amino acid modification by insertion of fatty acid at the alpha amino group of native amino acid or an epsilon amino group of a substituted lysine residue.

21. A peptide as claimed in claim 10, having at least one amino acid substitution and/or modification including N-glycated, N-alkylated, N-acetylated, N-acylated, N-isopropyl, and/or N-pyroglutamyl amino acids.

22. A pharmaceutical composition including at least one peptide according to claim 10 in admixture with a pharmaceutically acceptable excipient.

23. A pharmaceutical composition including at least one peptide according to claim 14 in admixture with a pharmaceutically acceptable excipient.

24. A pharmaceutical composition including at least one peptide according to claim 16 in admixture with a pharmaceutically acceptable excipient.

25. A pharmaceutical composition including at least one peptide according to claim 17 in admixture with a pharmaceutically acceptable excipient.

26. A pharmaceutical composition including at least one peptide according to claim 18 in admixture with a pharmaceutically acceptable excipient.

27. A pharmaceutical composition including at least one peptide according to claim 19 in admixture with a pharmaceutically acceptable excipient.

28. A pharmaceutical composition as claimed in claim 22 which further comprises at least one further pharmaceutically active agent, the, or each, further pharmaceutically active agent being selected from one or more sulphonylureas, meglitinides, metformin, and/or thiazolidinediones, or a mixture thereof.

29. A pharmaceutical composition as claimed in claim 22, the pharmaceutical composition being for delivery through transdermal, nasal inhalation, oral or injected routes.

30. A method for stimulating insulin secretion by activation of physiological stimulus-secretion coupling pathways, rather than by antimicrobial action involving cell lysis, the method comprising administering to an individual an effective amount of the peptide selected from the group comprising brevinins, dermaseptins, esculentins and peptides, or fragments thereof, as claimed in claim 10.

31. A method to stimulate insulin secretion and/or moderate blood glucose excursions, the method comprising administering to an individual an effective amount of a peptide selected from the group comprising brevinins, dermaseptins, esculentins and peptides, or fragments thereof, as claimed in claim 10.

32. A method to stimulate insulin secretion and/or moderate blood glucose excursions, the method comprising administering to an individual an effective amount of peptides, or fragments thereof, as claimed in claim 10.

33. A method to stimulate insulin secretion and/or moderate blood glucose excursions, the method comprising administering to an individual an effective amount of peptides, or fragments thereof, as claimed in claim 14.

34. A method to stimulate insulin secretion and/or moderate blood glucose excursions, the method comprising administering to an individual an effective amount of peptides, or fragments thereof, as claimed in claim 16.

35. A method to stimulate insulin secretion and/or moderate blood glucose excursions, the method comprising administering to an individual an effective amount of peptides, or fragments thereof, as claimed in claim 17.

36. A method to stimulate insulin secretion and/or moderate blood glucose excursions, the method comprising administering to an individual an effective amount of peptides, or fragments thereof, as claimed in claim 18.

37. A method to stimulate insulin secretion and/or moderate blood glucose excursions, the method comprising administering to an individual an effective amount of peptides, or fragments thereof, as claimed in claim 19.

38. A method for the treatment of type 1 or type 2 diabetes mellitus, the method comprising administering to an individual an effective amount of a peptide selected from the group comprising brevinins, dermaseptins, esculentins and peptides, or fragments thereof, as claimed in claim 10.
